(19) United States
(12) Patent Application Publication
(10) Pub. No.: US 2008/0000473 A1
(43) Pub. Date: Jan. 3, 2008

(54) pH-Based Methods and Devices for Preventing Hemagglutinin Cell Membrane Fusion

(76) Inventors: Hugo Stephenson, Durham, NC (US); Oren Cohen, Durham, NC (US); William D. Hare, Princeton, NJ (US)

Correspondence Address: WILLIAM DOUGLAS HARE, 3 ANDERSON LANE, PRINCETON, NJ, 08540, US

(21) Appl. No.: 11/617,821

(22) Filed: Dec. 29, 2006

Related U.S. Application Data
(60) Provisional application No. 60/766,039, filed on Dec. 29, 2005.

(51) Int. Cl. A61M 16/10; A61K 33/00; A61M 16/14; A61P 11/00; A61K 9/127

(52) U.S. Cl. 128/203.16; 424/450; 424/717

(57) ABSTRACT

The invention relates to devices and methods for treating or preventing avian flu, influenza and similar conditions using basic compounds, such as sodium bicarbonate, inhaled through inhalation devices such as nebulizers and the like. The invention also relates to processes for making the solution that is filled into the inhalation device. For example, the nebulizer can include a basic solution and at least a portion of the basic solution may be contained within one or both of a liposome and/or a micelle.

### RELATED APPLICATIONS

This application claims the benefit of prior U.S. Provisional Patent Application No. 60/766,039, filed on Dec. 29, 2005, the contents of which are incorporated in their entirety by reference.

### TECHNICAL FIELD OF THE INVENTION

The invention relates to devices and methods for treating or preventing avian flu, influenza and similar conditions using basic compounds, such as sodium bicarbonate, inhaled through inhalation devices such as nebulizers and the like. The invention also relates to processes for making the solution that is filled into the inhalation device.

### BACKGROUND OF THE INVENTION

Influenza viruses infect hundreds of millions of people every year, and result in numerous deaths—primarily clustered around the winter months. Influenza viruses are very simple in structure—consisting of a protein shell surrounding a tiny extract of genetic material. An infected cell inadvertently reads this extract of genetic material as its own, blindly following these instructions to build thousands of new viruses. In addition, the influenza virus has two surface glycoproteins that represent its main tools for infecting cells.

One of these glycoproteins is a neuraminase (NA), meaning that it breaks down neuraminic acid—an important component of sputum. The NA allows viruses to disperse through thick respiratory secretions and therefore infect more cells. Drugs such as Relenza® (zanamivir) and Tamiflu® (oseltamivir phosphate) inhibit the action of neuraminase, and therefore impair the ability of the virus to spread.

The second of these glycoproteins is a hemagluttinin, so named because it causes red cells to stick together when added to blood. Hemagluttinin is the influenza virus' principal means of infection, for this protein allows the virus to bind to sialic acid on a cell's surface, and mechanically inserts the genetic material into the cell.

The influenza virus infects a number of animals including humans, pigs and birds—and demonstrates the ability to create different combinations of H, N and protein shell subtypes, particularly when a cell is coinfected with different viruses. For example, a cell infected with both an H1N1 and H5N3 virus may create new H5N1 viruses. The H5N1 combination has proven to be highly lethal in birds, creating concern that exchanging the H5 protein into a human influenza virus could result in a disastrous pandemic.

The influenza A viruses associated with conditions such as avian flu are known to have hemagglutinin (HA) and neuraminidase (NA) membrane glycoproteins. The HA functions as the receptor binding and membrane fusion glycoprotein in cell entry and the NA functions as the receptor-destroying enzyme in virus release. According to some sources, there are fifteen subtypes of HA (H1-H15) that share between 40% and 60% sequence identity. Similarly, nine NA (N1-N9) subtypes have been identified with between 40% and 60% sequence identity. H1, H2, and/or H3 have been identified in humans in viruses causing pandemics in 1918, 1957, 1968, and 1977. Viruses containing H5 and H9 have been found in humans infected with influenza in Hong Kong and China in the late 1990's. Viruses containing all 15 HA subtypes have been found in avian species. Additional detail about the history, structure, and characteristics of the influenza virus are found at Ha et al., X-ray structure of H5 avian and H9 swine influenza virus hemagluttinins bound to avian and human receptor analogs (PNAS, vol. 98, no. 20, pp 11181-11186, Sep. 25, 2001, www.pnas.org/cgi/doi/10.1073/pnas.201401198); Ha et al., H5 avian and H9 swine influenza virus haemagglutinin structures: possible origins of influenza subtypes (The European Molecular Biology Organization Journal, Vol. 21, No. 5, pp 865-875 (2002)); Cross et al., Mechanism of Cell Entry by Influenza Virus (Expert Reviews in Molecular Medicine, pp 1-18, Aug. 6, 2001, http://www ermm.cbcu.cam.ac.uk/01003453h.htm); and Whittaker, Intracellular trafficking of influenza virus: clinical implications for molecular medicine (Expert Reviews in Molecular Medicine, Feb. 8, 2001, http://www ermm.cbcu.cam.ac.uk/01002447h.htm), the contents of each of which are incorporated herein by reference in their entirety.

It is stated that influenza viruses that infect humans or the genes that result in their HAs initially derive from avian viruses either directly or indirectly by cross-species infection or by gene re-assortment during mixed infections. Direct infections are believed to have occurred in the past. In 1997 and 1999, viruses from local birds are reported to have infected humans.

The HA portion of the virus has two functions, receptor binding to the cell and membrane fusion to the cell. The HA binding is similar in the avian virus and the human virus. The human virus prefers terminal sialic acids of glycoproteins and glycolipid receptors in alpha 2,6 linkage to galactose while the avian virus prefers alpha 2,3 linkages to galactose. The human lung and airway epithelial cells have an abundance of alpha 2,6 linkages. Moreover, the human lungs have potentially inhibitory mucins rich in alpha 2,3 linages. A paper by Ha et al. suggests that the receptor binding sites of human and avian HA differs with the human binding site being wider and favoring alpha 2,6 linkages while the avian binding site is narrower and favors alpha 2,3 linkages.

Reports in the scientific literature state that all fifteen HA subtypes fall within four major structural classes by correlating differences in the relative positions of subdomains in the H3, H5, and H9 HAs. Studies also suggest that there are some general mechanistic similarities in the low pH activation of membrane fusion, i.e., when the HA is exposed to a low pH, it is activated for membrane fusion—the molecule's second function in viral infection. In particular, there are ionizable residues that affect the pH at which the structural changes in HA occur that are required for membrane fusion. The wide distribution of these residues in the interfaces between all the structural interfaces that rearrange at low pH can result in destabilization of these interfaces that may affect the pH of refolding and that the trigger for refolding may be distributed rather than localized. Studies have also shown that three ionizable residues (His17 of HA1 and Asp109 and Asp112 of HA2) changed from solvent accessible to completely buried environments between the uncleaved and cleaved H3 subtype structures, raising the possibility that they may have a special role in triggering refolding.

Regarding the events occurring during membrane fusion, at least one study suggests that the HA2 residues 63, 87, and 88 make critical contacts in an intramolecular interface between the long alpha-helix, helix B, and the interhelical loop. During induction of the low pH conformation required for membrane fusion, these interactions are broken. It also is reported that the extensive changes in HA structure that is necessary for activating the HA-mediated membrane fusion at low pH or elevated temperature are irreversible and that premature activation result in the inactivation of infectivity.

As described above, the trafficking and processing steps that occur in cells that are infected with an influenza virus play a crucial role in the outcome of infection. The virus first binds to its host cell via specific sialic acid residues, which can control the species tropism of the virus. The internalization of the virus into the nucleus of the cell is dependent on a low pH, and this process is therapeutically targeted by the drug amantadine. Following replication, the newly formed viral genomes leave the nucleus and assemble into infectious particles at the plasma membrane. The targeting and processing of the various viral components at this late stage of the infectious cycle can have a major effect on the ability of the virus to spread and cause disease in its host. The release of viruses is dependent on the enzyme neuraminidase (NA), and this function has recently been targeted by the NA inhibitors, a new generation of drugs against influenza virus.

Because the hemagluttinin protein is so important to the influenza virus, it has evolved a number of protective features to evade body's defenses. First, it hides the sialic acid binding site from view within an external coat. Second, its external coat changes between generations in a process known as antigenic shift. By changing its external coat and hiding its important sialic acid binding site, the influenza virus prevents the body's defenses from recognizing the protein (and the influenza virus) on subsequent infections.

Pivotal to the influenza virus' success is the ability of the hemagluttinin protein to snap open at the right opportunity for infection. Without this opening mechanism, the virus would be unable to bind to cells or infect them with its genetic material. Research has shown that this opening mechanism is triggered by a drop in pH below 7.0. Since the body's natural response to infection involves neutrophil infiltration of the affected tissues and creation of various acids—this pH sensitive opening mechanism explains why infection can spread so rapidly in the presence of an immune response. In a way, the virus becomes stronger in response to our body's initial defense action as that defense reduces the pH.

Moreover, the pH sensitivity of the opening mechanism may also explain why influenza is more common in winter, in patients suffering from allergies, and in those who work in air conditioned environments. The body's defensive response to cold, dry air and/or allergens is the development of a thicker, more acidic respiratory mucus. Whereas the normal resting pH of lung fluids sits around 7.70, this drops below 7.0 as mucus gets thicker.

The scientific literature of end breath condensate (EBC) data demonstrates a decreased pH in response to cold, dry air, certain lung conditions, and allergens. For example, Vaughan et al., Exhaled Breath Condensate pH is a Robust and Reproducible Assay of Airway Acidity, (Eur Respir J 2003; 22:889-894), the contents of which are incorporated herein in its entirety by reference, report that exhaled breath condensate (EBC) pH is low in several lung diseases and that it normalizes with therapy. For example, the pH of EBC has been found to be low in stable asthma, chronic obstructive pulmonary disease (COPD), bronchiectasis, cystic fibrosis, and acute respiratory distress syndrome. The pH is markedly low in exacerbations of asthma, COPD and cystic fibrosis, with normalization of pH occurring with steroid or antibiotic therapy.

Altering the pH of the airway environment is known to affect multiple aspects of airway function. Nebulised citric or acetic acids are used to trigger bronchoconstriction and cough for testing antitussive agents. These effects are at least in part mediated by acid (protons) triggering capsaicin sensitive neurons and the release of tachykinins. Chlorine gas is thought to cause wheezing and coughing in substantial part because of the rapid formation of hydrochloric and hypochlorous acids upon contact with the airway lining fluid. In cats, instillation of 50 μL of 0.2 N hydrochloric acid into the trachea caused a 420% increase in airway resistance.

Multiple additional effects of airway acidification are expected. Mild acidification (below pH 6.5) increases mucous viscosity, converting it from sol to gel. Low environmental pH enhances inducible nitric oxide synthase 2 expression and activity in rat peritoneal macrophages through the action of tumour necrosis factor (TNF)-alpha and nuclear factor-kB.

Acidity affects airway cell protein expression. Guinea pig airway epithelial cells induce stress-related proteins including hsp72 in response to transient mild acidification. Low pH alters both the synthesis and secretion of TNF- by alveolar macrophages. Acidic insult augments hyperoxic injury in the rat, in part because of a loss of antioxidants. Acidification enhances oxidant and nitrative stresses by increasing reactivity of small inorganic molecules and by altering enzymatic antioxidant activities.

Kostikas et al. (in Am J Respir Crit Care Med. 2002; 165(10):1364-70 (ISSN: 1073-449X)), the contents of which are incorporated herein by reference, report on their study of pH in the expired breath condensate of patients with inflammatory airway diseases. Endogenous airway acidification, as assessed by pH in expired breath condensate, has been implicated in asthma pathophysiology. As such, the authors measured pH in breath condensate of patients with inflammatory airway diseases in stable condition and examined its relationship with the inflammatory process (as assessed by differential cell counts in induced sputum), oxidative stress (as assessed by H(2)O(2) and 8-isoprostane), and nitric oxide metabolism (as assessed by total nitrate/nitrite).

They found that the mean (95% confidence intervals) pH values were significantly lower in patients with chronic obstructive pulmonary disease (COPD) and bronchiectasis compared with patients with asthma and control subjects (7.16, 7.09-7.23 and 7.11, 7.04-7.19 versus 7.43, 7.35-7.52 and 7.57, 7.51-7.64, respectively, p<0.0001). They also report that patients with moderate asthma had significantly lower pH values compared with mild asthma and control subjects. In patients with COPD and bronchiectasis, the values of pH were significantly correlated with both sputum neutrophilia and oxidative stress. In patients with moderate asthma, a significant correlation was observed between pH and sputum eosinophilia, total nitrate/nitrite, and oxidative stress.

In Cáp et al. (in Allergy, 2005; 60(2):171-6 (ISSN: 0105-4538)), the contents of which are incorporated herein by reference, the authors analyzed exhaled leukotrienes (LT) in non-asthmatic adult patients with seasonal allergic rhinitis to determine whether LT levels in EBC were increased in non-asthmatic adult patients with seasonal allergic rhinitis both during and after the pollen season in comparison with healthy controls and to assess the changes of the LT levels after the pollen season. Using the knowledge that leukotrienes (LTs) are increased in exhaled breath condensate (EBC) in patients with asthma, the authors measured LT levels in non-asthmatic patients with seasonal allergic rhinitis.

The authors report that leukotriene concentrations (leukotrienes B(4), E(4) but not D(4)) were significantly increased in and after the pollen season in patients with seasonal allergic rhinitis in comparison with healthy controls. The values of all exhaled LTs were significantly decreased after the pollen season compared with the seasonal baseline. They were able to conclude that levels of exhaled LTB(4) and LTE(4) were higher in seasonal allergic rhinitis patients than in healthy controls and decreased after the pollen season as compared with levels in the pollen season. The seasonal allergic rhinitis patients with the highest in-season LT levels also had the post-season levels elevated, which may be an early marker of inflammatory process in the lower airways despite the absence of clinical symptoms of asthma.

### SUMMARY OF THE INVENTION

In one general aspect there is provided a method of treating or preventing a flu or influenza. The method includes administering a basic solution through an inhalation device.

Embodiments of the method may include one or more of the following features. For example, the basic solution may be free of any active pharmaceutical ingredient. The basic solution may be a bicarbonate solution. The bicarbonate solution may be sodium bicarbonate. The sodium bicarbonate solution may be present at a concentration of sodium bicarbonate between about 2% and 10% and, more narrowly, a concentration of sodium bicarbonate between about 2% and 3%.

The inhalation device may further include a second therapeutic agent. The second therapeutic agent may be one or more of a neuraminase inhibitor and a hemagluttinin inhibitor. The second therapeutic agent may be one or both of zanamivir and oseltamivir phosphate.

The inhalation device may be a nebulizer. The basic solution may be contained within a liposome and/or a micelle. The basic solution may be a bicarbonate solution. A first portion of the basic solution may be contained within a liposome and/or a micelle and a second portion of the basic solution may be exterior of the liposome and/or the micelle. The liposome and/or micelle may be configured to release the basic solution in a controlled release or delayed release manner.

The method may include treating or preventing a flu or influenza in a human. The treatment may occur along with administration of steroid and/or antibiotic therapy. It also may include treating or preventing a flu or influenza in livestock.

In another general aspect there is provided a nebulizer that includes a basic solution and at least a portion of the basic solution may be contained within one or both of a liposome and/or a micelle.

Embodiments of the nebulizer may include one or more of the following features. For example, a second portion of the basic solution may be exterior to the liposome and/or the micelle. The liposome and/or micelle may be configured to release the basic solution in a controlled release or delayed release manner. The basic solution may be a bicarbonate solution and the bicarbonate solution may be sodium bicarbonate. The sodium bicarbonate solution may be present at a concentration of sodium bicarbonate between about 2% and 10% or, more narrowly, at between about 2% and about 3%.

The nebulizer may further include a second therapeutic agent. The second therapeutic agent may include one or more of a neuraminase inhibitor and a hemagluttinin inhibitor. The second therapeutic agent may include one or both of zanamivir and oseltamivir phosphate.

In another general aspect there is provided a process for making an inhalation dosage form for treating or preventing a flu or influenza. The process includes (a) providing a first basic solution; and (b) filling an inhalation device with the basic solution.

Embodiments of the process may include one or more of the following features or those described above. For example, the basic solution may be incorporated within a liposome and/or a micelle. The basic solution comprises a sodium bicarbonate solution. The first basic solution and the second basic solution may be the same or may differ. The second basic solution may be a sodium bicarbonate solution. The inhalation device may be a nebulizer.

The process may further include (a) providing a second basic solution; (b) forming a liposome and/or a micelle containing the second basic solution; and (c) filling the liposome and/or the micelle into the inhalation device.

The process may further include providing a second therapeutic agent and the second therapeutic agent may be one or both of a neuraminase inhibitor and a hemagluttinin inhibitor. The second therapeutic agent may be one or both of zanamivir and oseltamivir phosphate.

The details of one or more embodiments of the invention are set forth in the description. Other features and advantages of will be apparent from the description and the claims.

### DESCRIPTION OF THE INVENTION

Focusing primarily on the hemagglutinin function, one method to treat or prevent avian flu is to block the function of hemagglutinin of fusing the virus to the membrane of the cell. As indicated above, there are similar mechanisms that appear to be present in the various hemagglutinins involving the low pH activation of membrane fusion, i.e., when the HA is exposed to a low pH, it is activated for membrane fusion by which the pH causes structural changes in HA that are required for membrane fusion. Accordingly, the inventors believe that they have developed methods to block the low pH activation of membrane fusion by generally increasing the pH of the environment in which the HA is activated. For example, in one embodiment, the pH is increased to a value above which activation can occur by administering a basic compound directly to the lungs. In this embodiment, a nebulizer containing, for example, a bicarbonate is used to orally administer the bicarbonate directly to the lungs and elevate the pH sufficiently to prevent the low pH activation of hemagluttinin. In this embodiment, the bicarbonate nebulizer may be used prophylactically or therapeutically. In modifications to this embodiment, the bicarbonate may by substituted by or included with additional compounds that elevate the pH in the lungs.

Nebulized sodium bicarbonate has been shown to safely provide symptomatic relief in patients exposed to chlorine, and it is probably useful with all irritant gases that liberate acid. For example, Bosse reports on the use of nebulized sodium bicarbonate in the treatment of chlorine gas inhalation (in J Toxicol Clin Toxicol. 1994; 32(3):233-41 (ISSN: 0731-3810)), the contents of which are incorporated in its entirety by reference. The article is a retrospective review of cases of chlorine gas inhalation in which the patients were treated with nebulized sodium bicarbonate. The patients were either exposed to chlorine producing acid/hypochlorite mixtures, chlorine gas in industrial settings, or chlorine gas in swimming pool settings. No patients in the study deteriorated clinically after nebulized sodium bicarbonate use. The authors conclude that nebulized sodium bicarbonate appears safe and merits prospective evaluation in the therapy of chlorine gas inhalation.

The nebulized sodium bicarbonate works by means of a neutralization reaction in which the damaging effect of the acid is limited. Any heat or gas generated by this process should be readily dissipated by the bronchopulmonary system. In treating chlorine gas exposure, some journal articles recommend that nebulized sodium bicarbonate be used in concentrations of less than 2% (which generally means about a 4:1 dilution of standard 8% sodium bicarbonate) while other articles report a use of 3.75%. A concentration much higher, e.g., 8-10%, may be suitable depending upon how much is administered. The inventors believe that a solution in this range is suitable, but that 2.4% is particularly suitable. However, the concentration of the bicarbonate can be increased as necessary, or in emergency situations, so long as the treatment of the flu is not compromised by lung damage. One source of sodium bicarbonate solution is Hospira Corporation, which has an ANDA approved for 7.5% and 8.4% concentrations of sodium bicarbonate solution for injection (ANDA No. 77-394). This solution may be diluted as necessary for making the solution for use in the inhalation device.

The treatment can be provided in a number of embodiments or with a number of objectives. For example, the objective of the treatment can be delivery of the bicarbonate for immediate treatment, for delayed treatment, for controlled treatment, or a combination of these. In particular, the nebulizer can be filled with a bicarbonate solution for immediate treatment or release and a bicarbonate solution in the form of a delayed release and/or controlled release. For example, in the controlled release or delayed release bicarbonate form, the bicarbonate solution can be incorporated into liposomes, micelles, or similar structures that release the bicarbonate over time or at a delayed time.

The liposome can be prepared by mixing a bicarbonate solution, a phospholipid (e.g., phosphatidyl choline, sphingomyelin or phosphatidyl ethanolamine, etc.), and then carrying out a known procedure (e.g., Ann. Rev. Biophys. Bioeng., Vol. 9, p. 467 (1980)), the contents of which are incorporated herein by reference, and as is known to one skilled in the relevant art. The liposome may contain other components as necessary to control its properties, such as release timing and quantity of the bicarbonate. For example, U.S. Pat. No. 4,911,928, the entirety of which is incorporated herein by reference, describes methods of making lipid vesicles by (a) forming a lipid phase of a surfactant and any lipid soluble materials to be incorporated into the lipid vesicles, (b) forming an oily phase of water immiscible oily material and an oil-suspendable or oil-soluble material to be incorporated; (c) forming a lipophilic phase by dispersing the oily phase in the lipid phase; (d) forming an aqueous phase of an aqueous solution and any aqueous soluble material to be incorporated into the lipid vesicles; and (e) blending the lipophilic phase and the aqueous phase under shear mixing conditions to thereby forming paucilamellar lipid vesicles having about 2-8 exterior lipid bilayers surrounding a substantially amorphous center with the water immiscible oily material substantially filling the amorphous central cavity.

The surfactant is described as being selected from polyoxyethylene fatty esters, polyoxyethylene fatty acid ethers, diethanolamides, long chain acyl hexosamides, long chain acyl amino acid amides, long chain acyl amides, polyoxyethylene (20) sorbitan mono- or trioletate; polyoxyethylene glyceryl monosterate with 1-10 polyoxyethylene groups; and glycerol monosterate; The patent notes that the lipophilic phase may further include a steroid and the steroid may be a sterol selected from cholesterol, hydrocortisone, and analogues and derivatives thereof. It also notes that the lipophilic phase may further include a charge-producing agent and the charge-producing agent may have a negative charge-producing agent selected from oleic acid, dicetyl phosphate, cetyl sulphate, phosphatidic acid, phosphatidyl serine, and mixtures thereof. The process may be modified as necessary to include a bicarbonate solution

The micelles can be prepared by mixing a bicarbonate solution with a surface active agent (e.g., a polyoxysorbitan fatty acid ester or a salt of a fatty acid, etc.), and then carrying out a known procedure to form a micelle, as is known to one skilled in the relevant art. For example, U.S. Pat. No. 5,629,021, the entirety of which is incorporated herein by reference, describes methods of making micellular nanoparticles for delivering drugs. The micelles are described as having a diameter of between about 25 and 1000 nm, and are formed with a lipophilic phase that includes an oil, a stabilizer and an alcohol-based initiator, and hydrated with an aqueous solution. The stabilizer is described as being selected from the group consisting of Tween 60, Tween 80, nonylphenol polyethylene glycol ethers, and mixtures of these, the initiator is described as being selected from the group consisting of alcoholic materials containing methanol, ethanol and mixtures of these, and the oil is described as being selected from the group consisting vegetable oils, nut oils, fish oils, lard oil, mineral oils, squalane, tricaprylin, and mixtures of these. In one embodiment of the present invention, the aqueous solution includes the bicarbonate.

Once the liposomes and/or micelles are formed with the bicarbonate solution, the liposomes and/or micelles are mixed with a solution of bicarbonate and filled into a nebulizer. The nebulizer is then packaged and ready for patient use. Alternatively, to form the administered bicarbonate in a single step, a molar excess of the bicarbonate solution is mixed with the components of the micelles and/or liposomes. In this manner, the liposomes and/or micelles will be formed with the limited amount of their constituent materials and an excess of the bicarbonate solution will remain. This combined liposome/micelle and bicarbonate solution then is filled into the nebulizer and ultimately provided to the patient.

In patient use, there are a number of options. A first simple option is the bicarbonate solution filled into a nebulizer. This option provides immediate availability of the bicarbonate solution to treat the avian flu because there is nothing in the dosage form to prevent the bicarbonate from affecting the pH. In this treatment mode, however, the patient is required to use the nebulizer periodically to maintain a basic pH in the lungs as the body's pH buffering mechanisms return the pH to its normal value. The concentration of the bicarbonate solution can be varied, for example, based on the desired amount of solution to be administered with each use of the nebulizer.

A second option is the nebulizer containing a bicarbonate solution in a liposome or micelle. When this is administered to a patient, this option provides a controlled release or delayed release of the bicarbonate solution over time and thereby provides a different availability of the bicarbonate to the lungs, both in timing and amount. This mode, however, may not provide immediate increase in pH to the patient's lungs as believed necessary to prevent the hemagluttinin opening mechanism. It will provide a long term maintenance program to keep a basic pH and with less frequent use of the nebulizer as compared to the first option. The concentration of the bicarbonate in the liposomes/micelles can be varied based on the release rate of the bicarbonate solution into the lungs.

A third, more complex option, is a combination of the first and second options. This can be in the form of two nebulizers, the immediate availability version of option one and the delayed or controlled availability version of option two. It also can be in the form of a single nebulizer containing the bicarbonate solution and the liposomes/micelles containing a bicarbonate solution. This second alternative offers patient convenience with respect to dosing but requires manufacture of a more complex solution for filling into the nebulizer. However, it is believed to be well within the skill of one of ordinary skill in the art. For example, the amount of bicarbonate solution can be increased beyond that which will be used in making the micelles such that the excess bicarbonate solution to provide immediate availability. The amount of excess bicarbonate solution can be varied depending on the amount desired to be immediately available.

In each of these embodiments that include a nebulizer, it should be understood that additional therapeutic agents may be used with the bicarbonate. For example, it is possible that by incorporating a compound such as Relenza® (zanamivir) and Tamiflu® (oseltamivir phosphate) in the nebulizer with the bicarbonate solution, the actions of both neuraminase and the hemagluttinin can be inhibited. Of course, the additional therapeutic agent can be administered in a separate dosage form and merely taken along with the use of the nebulized bicarbonate. It also should be understood that the bicarbonate can be administered as a prophylactic or as a therapeutic agent. The treatments described herein are believed to be suitable to any flu or influenza, such as avian flu, that involves a neuraminase and/or a hemagluttinin.

Similarly, it should be understood that the bicarbonate can be in the form of a solid as well as a solution and can be in several forms, such as sodium bicarbonate, potassium bicarbonate, etc. The key characteristic that is needed is the ability to increase the pH in the lungs, either immediately or over time, when administered. Moreover, an acid-base buffer can be used in addition to or in place of the bicarbonate to ultimately provide the increased pH in the lungs. Further, as another treatment form, the invention includes administering a gas that creates an increase in pH in the lungs (similar to ammonia, etc.) but yet is safe in the administered quantities. For example, the patient can take a dosage form that releases a basic vapor for breathing in the lungs. The dosage form, besides being a gas, can be in the form of an orally administered form such as a candy or other long-lasting oral candy—cough drops, menthol drops, etc.—that when dissolved slowly in the mouth, provide a vapor or gas that causes a basic environment in the lungs when breathed into the lungs. The basic gas can even be scented, such as incense or in a wax as a scented candle that provides a basic smoke which is breathed in as a therapy or prophylactic.

The inhalation device used herein may be one of numerous inhalation type devices, including nebulizers. Any nebulizer is contemplated as being useful for the methods provided herein. In particular, the nebulizers for use herein nebulize liquid formulations. The nebulizer may produce the nebulized mist by any method known to those of skill in the art, including, but not limited to, compressed air, ultrasonic waves, or vibration. The nebulizer may further have an internal baffle. The internal baffle, together with the housing of the nebulizer, selectively removes large droplets from the mist by impaction and allows the droplets to return to the reservoir. The fine aerosol droplets thus produced are entrained into the lung by the inhaling air/oxygen. U.S. Pat. No. 6,667,344 provides details of nebulizers and their formulation for delivering pharmaceutical agents. The contents of this patent are incorporated herein by reference.

Nebulizers that nebulize liquid formulations include those from the following sources: Pari GmbH (Starnberg, Germany), DeVilbiss Healthcare (Heston, Middlesex, UK), Healthdyne, Vital Signs, Baxter, Allied Health Care, Invacare, Hudson, Omron, Bremed, AirSep, Luminscope, Medisana, Siemens, Aerogen, Mountain Medical, Aerosol Medical Ltd. (Colchester, Essex, UK), AFP Medical (Rugby, Warwickshire, UK), Bard Ltd. (Sunderland, UK), Carri-Med Ltd. (Dorking, UK), Plaem Nuiva (Brescia, Italy), Henleys Medical Supplies (London, UK), Intersurgical (Berkshire, UK), Lifecare Hospital Supplies (Leies, UK), Medic-Aid Ltd. (West Sussex, UK), Medix Ltd. (Essex, UK), Sinclair Medical Ltd. (Surrey, UK), and many others.

Nebulizers for use herein include, but are not limited to, jet nebulizers (optionally sold with compressors), ultrosonic nebulizers, and others. Exemplary jet nebulizers for use herein include Pari LC plus/ProNeb, Pari LC plus/ProNeb Turbo, Pari LC plus/Dura Neb 1000 & 2000, Pari LC plus/Walkhaler, Pari LC plus/Pari Master, Pari LC star, Omron CompAir XL Portable Nebulizer System (NE-C18 and JetAir Disposable nebulizer), Omron CompAir Elite Compressor Nebulizer System (NE-C21 and Elite Air Reusable Nebilizer), Pari LC Plus or Pari LC Star nebulizer with Proneb Ultra compressor, Pulmo-aide, Pulmo-aide LT, Pulmo-aide traveler, Invacare Passport, Inspiration Healthdyne 626, Pulmo-Neb Traverler, DeVilbiss 646, Whisper Jet, Acorn II, Misty-Neb, Allied aerosol, Schuco Home Care, Lexan Plasic Pocet Neb, SideStream Hand Held Neb, Mobil Mist, Up-Draft, Up-Draft II, T Up-Draft, ISO-NEB, AVA-NEB, Micro Mist, and PulmoMate. Exemplary ultrasonic nebulizers for use herein include MicroAir, UltraAir, Siemens Ultra Nebulizer 145, CompAir, Pulmosonic, Scout, 5003 Ultrasonic Neb, 5110 Ultrasonic Neb, 5004 Desk Ultrasonic Nebulizer, Mystique Ultrasonic, Luminscope's Ultrasonic Nebulizer, Medisana Ultrasonic Nebulizer, Microstat Ultrasonic Nebulizer, and MABISMist Hand Held Ultrasonic Nebulizer. Other nebulizers for use herein include 5000 Electromagnetic Neb, 5001 Electromagnetic Neb 5002 Rotary Piston Neb, Lumineb I Piston Nebulizer 5500, Aeroneb.TM. Portable Nebulizer System, Aerodose.TM. Inhaler, and AeroEclipse Breath Actuated Nebulizer.

The pharmaceutical compositions used in the nebulizer or other inhalation device are sterile filtered and filled in vials, including unit dose vials providing sterile unit dose formulations which are used in a nebulizer and suitably nebulized. Each unit dose vial is sterile and is suitably nebulized without contaminating other vials or the next dose. The unit dose vials may be formed in a form-fill-seal machine or by any other suitable method known to those of skill in the art. The vials may be made of plastic materials that are suitably used in these processes. For example, plastic materials for preparing the unit dose vials include, but are not limited to, low density polyethylene, high density polyethylene, polypropylene and polyesters. In one embodiment, the plastic material is low density polyethylene.

The compositions provided herein optionally may include excipients and additives. The particular excipient or additive for use in the compositions for long term storage provided herein may be determined empirically using methods well known to those of skill in the art. Excipients and additives are any pharmacologically suitable and therapeutically useful substance which is not an active substance. Excipients and additives generally have no pharmacological activity, or at least no undesirable pharmacological activity. The excipients and additives include, but are not limited to, surfactants, stabilizers, complexing agents, antioxidants, or preservatives to prolong the duration of use of the finished formulation, flavorings, vitamins, or other additives known in the art. Preservatives include, but are not limited to, those that protect the solution from contamination with pathogenic particles. Antioxidants include, but are not limited to, vitamins, provitamins, ascorbic acid, vitamin E or salts or esters thereof. The excipient should not counteract the objective of the device by lowering the pH such that the method of treating is not useful or beneficial.

The nebulizer can provide the bicarbonate either heated, cooled, and/or humidified. Separately or with the nebulizer, the patient can be provided a temperature controlled air supplier for breathing in heated or cooled air. Similarly, although the term nebulizer is used throughout herein, other suitable inhalation devices may used. Because pH has been found to be reduced in cold dry air, it is expected that the use of a heated, humidified air supplier may be of use and, similarly, that a heated nebulizer of a bicarbonate solution will be of use.

While several particular forms of the invention have been described and exemplified, it will be apparent that various modifications and combinations of the invention detailed herein can be made without departing from the spirit and scope of the invention. For example, another use of the nebulized basic or bicarbonate solution is to treat avian flu or influenza in livestock, such as chickens, cows, and pigs, to prevent the spread of, e.g., avian flu amongst livestock. The nebulizer can be of industrial strength and industrial fabrication materials and used to provide a nebulized solution to an entire room and/or individual livestock. Accordingly, it is not intended that the invention be limited, except as by the appended claims.

We claim:

1. A method of treating or preventing a flu or influenza, the method comprising administering a basic solution through an inhalation device.

2. The method of claim 1, wherein the basic solution comprises a bicarbonate solution.

3. The method of claim 2, wherein the bicarbonate solution comprises sodium bicarbonate.

4. The method of claim 3, wherein the sodium bicarbonate solution comprises a concentration of sodium bicarbonate between about 2% and 10%.

5. The method of claim 4, wherein the sodium bicarbonate solution comprises a concentration of sodium bicarbonate between about 2% and 3%.

6. The method of claim 1, wherein the inhalation device comprises a nebulizer.

7. The method of claim 1, wherein the inhalation device further comprises a second therapeutic agent.

8. The method of claim 7, wherein the second therapeutic agent comprises one or both of a neuraminase inhibitor and a hemagluttinin inhibitor.

9. The method of claim 7, wherein the second therapeutic agent comprises one or both of zanamivir and oseltamivir phosphate.

10. The method of claim 1, wherein the basic solution is contained within a liposome and/or a micelle.

11. The method of claim 10, wherein the basic solution comprises a bicarbonate solution.

12. The method of claim 1, wherein a first portion of the basic solution is contained within a liposome and/or a micelle and a second portion of the basic solution is exterior of the liposome and/or the micelle.

13. The method of claim 12, wherein the basic solution comprises a bicarbonate solution.

14. The method of claim 12, wherein the liposome and/or micelle is configured to release the basic solution in a controlled release or delayed release manner.

15. The method of claim 1, wherein the method comprising treating or preventing a flu or influenza in a human.

16. The method of claim 1, wherein the method comprising treating or preventing a flu or influenza in livestock.

17. A nebulizer comprising a basic solution, wherein at least a portion of the basic solution is contained within one or both of a liposome and/or a micelle.

18. The nebulizer of claim 17, wherein a second portion of the basic solution is exterior to the liposome and/or the micelle.

19. The nebulizer of claim 17, wherein the liposome and/or micelle is configured to release the basic solution in a controlled release or delayed release manner.

20. The nebulizer of claim 17, wherein the basic solution comprises a bicarbonate solution.

21. The nebulizer of claim 20, wherein the bicarbonate solution comprises sodium bicarbonate.

22. The nebulizer of claim 21, wherein the sodium bicarbonate solution comprises a concentration of sodium bicarbonate between about 2% and 10%.

23. The nebulizer of claim 22, wherein the sodium bicarbonate solution comprises a concentration of sodium bicarbonate between about 2% and 3%.

24. The nebulizer of claim 17, wherein the nebulizer further comprises a second therapeutic agent.

25. The nebulizer of claim 24, wherein the second therapeutic agent comprises one or both of a neuraminase inhibitor and a hemagluttinin inhibitor.

26. The nebulizer of claim 24, wherein the second therapeutic agent comprises one or both of zanamivir and oseltamivir phosphate.

27. A process for making an inhalation dosage form for treating or preventing a flu or influenza, the process comprising:
(a) providing a first basic solution; and
(b) filling an inhalation device with the basic solution.

28. The process of claim 27, wherein the basic solution is incorporated within a liposome and/or a micelle.

29. The process of claim 27, further comprising:
(a) providing a second basic solution;
(b) forming a liposome and/or a micelle containing the second basic solution; and
(c) filling the liposome and/or the micelle into the inhalation device.

30. The process of claim 27, wherein the inhalation device comprises a nebulizer.

31. The process of claim 27, wherein the basic solution comprises a sodium bicarbonate solution.

32. The process of claim 29, wherein the first basic solution and the second basic solution are the same.

33. The process of claim 29, wherein the first basic solution and the second basic solution differ.

34. The process of claim 29, wherein the second basic solution comprises a sodium bicarbonate solution.

35. The process of claim 27, further comprising providing a second therapeutic agent.

36. The process of claim 35, wherein the second therapeutic agent comprises one or more of a neuraminase inhibitor and a hemagluttinin inhibitor.

37. The process of claim 36, wherein the second therapeutic agent comprises one or both of zanamivir and oseltamivir phosphate.
